(19) United States
(12) Patent Application Publication
(10) Pub. No.: US 2021/0059955 A1
(43) Pub. Date: Mar. 4, 2021

(54) SYSTEMS AND METHODS FOR DECRYSTALLIZATION OF URIC ACID

(71) Applicant: RL Patents, LLC, San Francisco, CA (US)

(72) Inventor: Ricky Lam, San Francisco, CA (US)

(21) Appl. No.: 17/000,450

(22) Filed: Aug. 24, 2020

Related U.S. Application Data
(60) Provisional application No. 62/891,596, filed on Aug. 26, 2019.

(51) Int. Cl. A61K 9/70; A61K 33/00

(52) U.S. Cl. CPC A61K 9/7046; A61K 33/00

(57) ABSTRACT

Systems and methods for treatment of hyperuricemia through the application of a base compound, such as, e.g., an antacid or alkali, are presented. Examples of an antacid or alkali includes, e.g., calcium carbonate, aluminum hydroxide, magnesium hydroxide, and sodium bicarbonate. The application uses a transdermal patch for preventing, minimizing or reversing hyperuricemia. The active ingredient or agent, e.g. antacid or alkali, may be distributed in the patch in any suitable manner. For example, a specified amount of the active ingredient or agent may be contained in a single drug-containing layer or multiple drug-containing layers. In addition to the active ingredient or agent, the drug-containing layer may comprise a carrier material for carrying the active ingredient or agent. The active ingredient or agent may be mixed with the carrier material, such as, e.g. homogenously blended, heterogeneously dispersed, encapsulated within, or any combination thereof.

### CLAIMS OF PRIORITY

This patent application is a continuation-in-part and claims priority from:
(1) U.S. provisional patent application No. 62/891,596, entitled ‘Methods and systems for decrystallization of uric acid’, filed Aug. 26, 2020.

### FIELD OF TECHNOLOGY

This disclosure relates generally to techniques for application of an active ingredient or agent in the field of medicine.

### BACKGROUND

Hyperuricemia is a disease caused by an increased level of uric acid in blood resulting from metabolic disorder of purine in the human body. The production and excretion of uric acid in vivo are approximately in equal amounts. About one third of the production amount is derived from foods, and two thirds is synthesized by the body; while one third of the excretion amount is through the intestinal tracts, and two thirds is dispensed from the kidneys. Uric acid concentration may increase for two reasons. The first is the reduced urinary excretion of uric acid, and the second one is the enhanced biosynthesis of uric acid due to regulation disorders.

Uric acid content in blood plasma of healthy people is approximately 2.0-6.0 mg/dL. When it exceeds 6.8 mg/dL, monosodium urate (MSU) crystals may be deposited at parts of the human body and may lead to inflammatory arthritis, namely, “gout”. Gout is a common condition, in which crystals precipitate within joints and soft tissues and elicit an inflammatory response. When the condition progresses, the crystals may accumulate and form subcutaneous lumps called “tophi”. If gout is not prevented or treated properly, the disease may deteriorate the affected joints and the attacks may occur more frequently. Repeated occurrences will cause permanent damages to the joints, including long-term pain and stiffness, limited mobility and joint deformity.

Acute gout flares may come on suddenly, and go away after five to 10 days, and can keep recurring. Several studies suggest that MSU crystals may drive the generation of crystal-specific antibodies that facilitate future MSU crystallization. Current knowledge suggests that the intense joint inflammation occurs as white blood cells engulf the uric acid crystals, causing pain, heat, and redness of the joint tissues. Gouty arthritis is due to MSU-crystal-induced release of proinflammatory cytokines from leukocytes. Among the many cytokines implicated, IL-1 may have a special role in the inflammatory network, as MSU crystals stimulate IL-1 release by monocytes and synovial mononuclear cells.

The natural progression and clinical manifestations of gout includes an asymptomatic hyperuricemia state followed by an onset of acute gouty arthritis, an intermission period, and then a chronic arthritis of gouty tophus. Although the presence of hyperuricemia is essential for the formation of crystals, only a fraction of hyperuricemic patients develop gout. In most patients, the gout outbreak is related to the changing—increasing or decreasing—rate of blood uric acid level, relevant to its stable level. Local factors have been hypothesized to also play a role in crystal formation, such as, e.g., temperature, pH, mechanical stress, cartilage components, and other synovial and serum factors. Hyperuricemia can be congenital or an acquired defect, and prevalence appears to increase with age, particularly in men, while fewer is seen in women until after menopause. Case reports have shown it to be related with family history, primarily autosomal recessive inheritances.

At present, there are limited types of anti-gout agents. For acute onset, colchicine, non-steroidal anti-inflammatory agents and hormones are mainly used. Clinical treatment of gout involves the administration of agents inhibiting uric acid synthesis, e.g., allopurinol and febuxostat, agents promoting uric acid excretion, e.g., probenecid, sulfinpyrazone, benzbromarone, Lesinurad, non-steroidal anti-inflammatory agents and hormones. However, these agents are not satisfactory for the treatment of gout because of poor efficacy and numerous adverse side effects. Thus, there is a need for new agent(s) to replace or complement available pharmaceutical approaches.

### SUMMARY

Systems and methods for treatment of hyperuricemia through the application of a base compound, such as, e.g., an antacid or alkali, are presented. A base may be defined as a substance that accepts hydrogen ions and has a value on the pH-scale that is greater than seven. Examples of an antacid or alkali includes, e.g., calcium carbonate, aluminum hydroxide, magnesium hydroxide, and sodium bicarbonate.

The application uses a transdermal patch for preventing, minimizing or reversing hyperuricemia. The active ingredient or agent, e.g. antacid or alkali, may be distributed in the patch in any suitable manner. For example, a specified amount of the active ingredient or agent may be contained in a single drug-containing layer or multiple drug-containing layers. In addition to the active ingredient or agent, the drug-containing layer may comprise a carrier material for carrying the active ingredient or agent. The active ingredient or agent may be mixed with the carrier material, such as, e.g. homogenously blended, heterogeneously dispersed, encapsulated within, or any combination thereof.

The transdermal patch may include any of a variety of design types, including but not limited to drug-in-adhesive, reservoir, matrix and multi-laminate. Drug-in-adhesive patches may compise an adhesive layer containing the active ingredient or agent. In this type of patch, the adhesive layer not only serves to hold the various layers intact while adhering to the skin, but it is also responsible for storing and releasing the active ingredient or agent. The adhesive layer may be surrounded by a temporary liner and a backing. The reservoir patch may comprise a reservoir separated from the skin by an inert polymeric membrane that controls the rate at which the drug is delivered. These patches offer a benefit that as long as the active ingredient or agent solution in the reservoir remains saturated, the drug release rate through the membrane is constant. The matrix patch is also known as a monolithic device. The matrix system incorporates a backing layer, a matrix layer, and an adhesive layer. The matrix layer is made of a polymer material in which the active ingredient or agent is distributed, and the rate at which the drug is released from the device is controlled by the polymer matrix. With this type of system, the release rate of active ingredient or agent falls off with time as the active ingredient or agent in the skin-contacting side of the matrix is depleted. Lastly, the multi-laminate patch may include two or more adhesive layers containing the active ingredient or agent with different release times. A bolus dose may initially deploy, followed by a maintenance dose. The active ingredients or agents in each of the adhesive layers may be the same or different.

### BRIEF DESCRIPTION OF THE DRAWINGS

Example embodiments are illustrated by way of example and are not limited to the figures of the accompanying drawings, in which, like references indicate similar elements.

FIG. 1 is a schematic diagram of the physical chemistry of uric acid and monosodium urate formation.

FIG. 2 is a model of the initiation and propagation of monosodium urate crystallization.

FIGS. 3A-D show different configurations for a transdermal patch of the present invention.

FIG. 4 is a flowchart of an example method for manufacturing a drug-in-adhesive patch.

FIG. 5 is a flowchart of an example method for manufacturing a reservoir patch.

FIG. 6 is a flowchart of an example method for manufacturing a matrix patch.

FIGS. 7A-B are flowcharts of an example method for manufacturing a multilaminate patch.

FIG. 8 is a flowchart of a method for using the transdermal patch.

FIG. 9 is a flowchart of another method for using the transdermal patch.

FIG. 10 is a graphical representation of Table 1.

### DETAILED DESCRIPTION

Although the present has been described with reference to specific examples, it will be evident that various modifications and changes may be made without departing from the broader spirit and scope of the various examples. The modifications and variations include any relevant combination of the disclosed features. In addition, the components shown in the figures, their connections, couplings, relationships, and their functions, are meant to be exemplary only, and are not meant to limit the examples described herein.

Systems and methods for treatment of hyperuricemia through the application of a base compound, such as, e.g., an antacid or alkali, are presented. A base may be defined as a substance that accepts hydrogen ions and has a pH greater than seven. Examples of an antacid or alkali includes, e.g., calcium carbonate, aluminum hydroxide, magnesium hydroxide, and sodium bicarbonate.

Sodium bicarbonate or sodium hydrogen carbonate, commonly known as “baking soda”, is a chemical compound with the formula NaHCO₃. It has a molar mass of 84.0066 g/mol and often appears as a fine powder. Sodium bicarbonate is a well understood salt that breaks down in fluids, including blood and urine, to form sodium cation (Nat) and a bicarbonate anion (HCO₃⁻). This breakdown makes the fluid alkaline, meaning it may be able to neutralize acid. HCO₃⁻ is the conjugate base of carbonic acid, and hence immediately forms carbonic acid by reacting with H+ due to hydrolysis. The fluid now contains excess of OH⁻ ions leading to its basic nature. Oral uses of the compound include treatment for metabolic acidosis, bowel cleansing, poor kidney function, indigestion, increasing exercise performance, high potassium in the blood, reviving newborns, and stomach ulcers; topical uses include treatment for chemical burns, dental plaque, earwax removal, eczema, insect bites or stings, inflammation in the mucous membranes lining the digestive tract, poison oak and poison ivy, pruritus, e.g., itchy skin, and scaly, and psoriasis, e.g., itchy skin; intravenous (IV) uses include treatment for severe metabolic acidosis, heart resuscitation, poor kidney function, cocaine toxicity, prevention of kidney damage caused by dyes used during some X-ray exams, poisoning from certain allergy medications, reviving newborns, pesticide poisoning, prevention of chemotherapy side effects, breakdown of muscles, and fluid build-up in the lungs caused by chemical. In addition to the therapeutic usages just mentioned, sodium bicarbonate can be used as a food or beverage additive for human consumption. As such, it is generally regarded as a safe chemical, and has a low risk of toxicity in humans.

A novel medical application of sodium bicarbonate is presented. As previously mentioned and is well understood, the breakdown of sodium bicarbonate creates an alkaline environment that is able to neutralize one or more acids, while the main cause of hyperuricemia is an elevated concentration of uric acid. Uric acid, the final product of purine metabolism, is a weak acid that circulates as the deprotonated urate anion under physiologic conditions, and combines with sodium ions to form MSU crystals, which are known to have a triclinic structure, in which stacked sheets of purine rings form needle-shaped crystals that are observed microscopically. If sodium bicarbonate is released to the environment, it will absorb moisture from the surroundings and form lumps; in water, it will break down into carbon dioxide and sodium carbonate. In dry air, sodium bicarbonate does not break down. In the presence of an acid, such as uric acid, sodium bicarbonate may act as a base and forms carbonic acid, which readily decomposes into carbon dioxide and water. Carbon dioxide and water may be easily extreted by the human body. In addition, sodium bicarbonate may interact with other acids present in the body, such as, e.g., lactic acid, and may increase overall pH and thus may drive equilibrium of uric acid away from combining with another particle, such as, e.g., sodium, to form monosodium urate crystals.

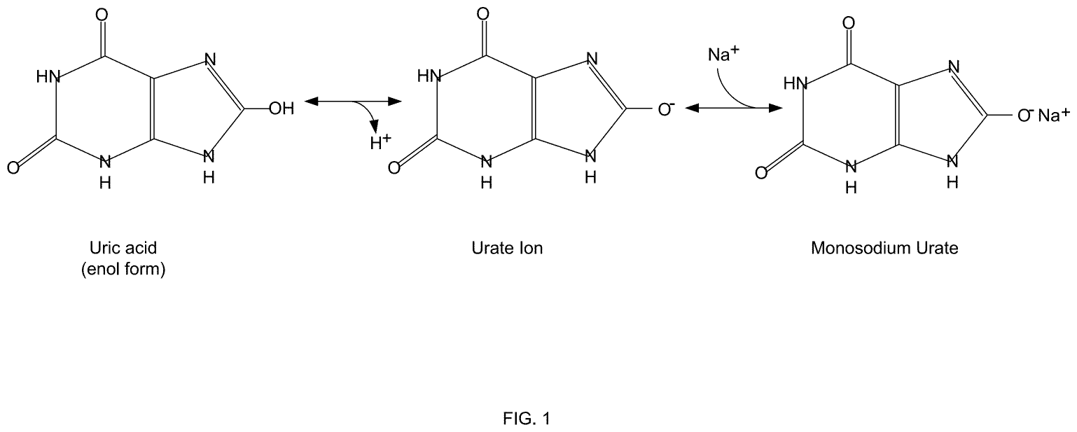

FIG. 1 is a schematic diagram of the physical chemistry of uric acid and monosodium urate formation. Uric acid is a weak, hydrogenated organic acid. At physiological conditions, such as, e.g., pH 7.4 and 37° C., chemical equilibrium may lean to the right towards formation of urate, which circulates in plasma and synovial fluid in a monodeprotonated ionic form. Urate ions can combine with sodium present in solution to form the less-soluble salt form, monosodium urate. In the crystalline state, urate is observed in its fully protonated acid form, e.g., uric acid, or as a variety of salts formed by deprotonated or partially deprotonated urate. Monosodium urate monohydrate (NaC₅H₃N₄O₃.H₂O), in which a urate molecule is bonded to one sodium and one water molecule, is one of the most common forms of crystallized urate and comprises the primary deposits seen in gouty arthritis, although urate precipitation with other mineral phases may also be possible.

Presented is the use of an antacid or alkali, such as, e.g., sodium bicarbonate, to shift chemical equilibrium towards the dissociation of monosodium urate crystals by neutralizing acids found in bodily fluids, such as, e.g., lactic acid and uric acid, as previously discussed in detail, of a hyperuricemic patient. This would effectively lower overall acidity of the blood serum and synovial fluid of the patient. This lowered acidity level may continue to drive the equilibrium of its dissociation equation towards further dissolution of monosodium urate crystals. As such, hyperuricemia may be effectively reversed.

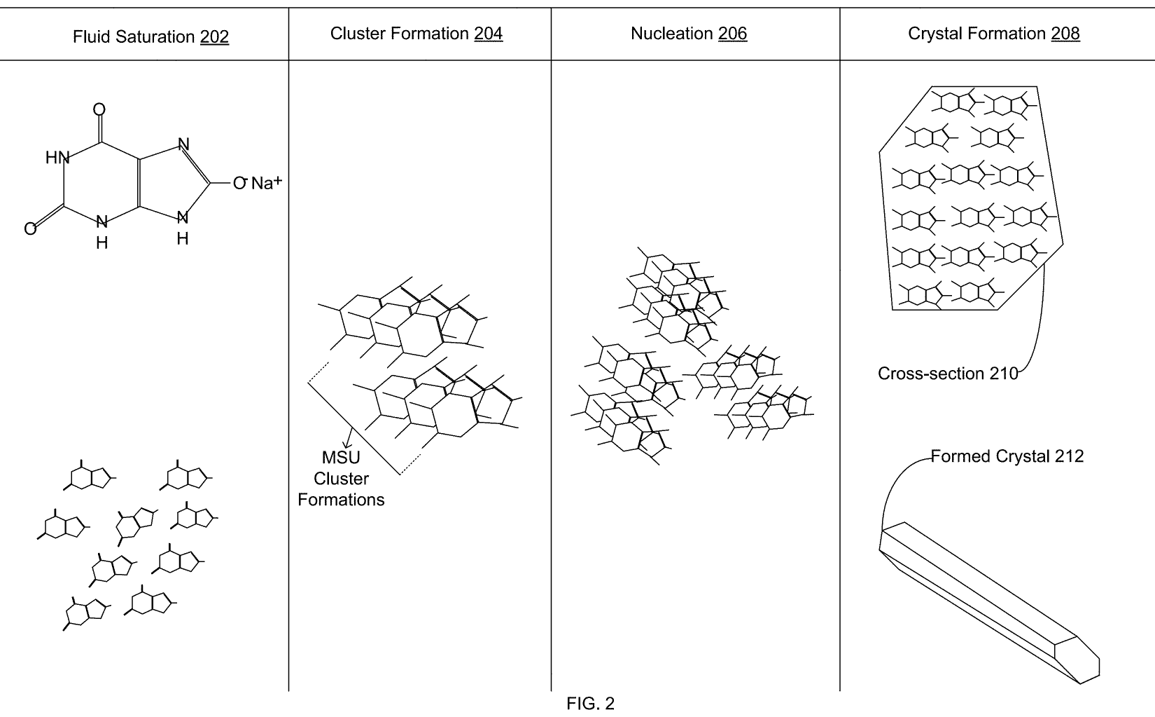

FIG. 2 is a model of the initiation and propagation of monosodium urate crystallization. At fluid saturation 202, monosodium urate molecules remain fully in solution until an event that changes their solubility, such as, e.g., increased concentration and/or decreased temperature. At cluster formation 204, soluble monosodium urate molecules begin to cluster while still in solution. At nucleation 206, clusters begin to aggregate into crystal nuclei, the basis for additional crystal formation and growth. Formation of fully-aggregated monosodium urate crystals occur at crystal formation 208. Monosodium urate molecules in the crystals are arranged in flat sheets as seen in cross-section 210. The surfaces of these flat sheets may form the major growth faces of the crystals, with the flat sheets stacking along the crystal's long axis. Fully formed crystal 212 may comprise three axes that are non-perpendicular, e.g., a triclinic structure. The formed crystal 212 may further grow in size.

Temperature is an environmental factor that may play a role in MSU crystal formation through effects on urate solubility. In vitro studies performed in aqueous solutions suggest that a reduction of even 2° C., e.g., from 37 to 35° C., may be sufficient to lower the solubility point of urate from 6.8 to 6.0 mg/dL. This response to temperature may explain, in-part, why the first metatarsophalangeal joint—an area of relatively reduced perfusion, which suggests reduced heat delivery from the body core, and increased surface-to-volume ratio promoting heat radiation and loss—is the most common site for first gouty attacks. Conversely, the heat provoked by the inflammatory gouty attack, due to increased perfusion and tissue metabolism of the affected joint, may contribute to the subsequent dissolution of crystals, and to the observation that gout attacks may be self-limiting.

Similar to cold temperature, the presence of an acidic environment may also facilitate MSU crystallization. The reduction of pH promotes MSU nucleation in in-vitro systems, independent of its solubility level, has been observed, although the mechanism is not yet well understood. In addition, pH exerts an indirect effect on MSU crystallization by increasing calcium ion concentrations that consequently reduces MSU solubility and promotes nucleation. The increased levels of free calcium ions in the setting of lower pH resulting from displacement of plasma protein-bound calcium into the liquid phase may explain the relationship between acidic environments and MSU nucleation. The initial nucleus of an MSU crystal may be a calcium urate molecule, or that, because of their nearly identical atomic radii, calcium ions may substitute for sodium in the urate crystal lattice.

Acidosis occurs in conditions such as strenuous exercise, respiratory insufficiency, and ethanol consumption, all of which are associated with the development of gout attacks. Since systemic acidosis may also promote decreased renal urate excretion, the resulting increases in systemic hyperuricemia may also contribute to the risk for crystallization in conditions of acidosis. The metabolic activity of neutrophils during phagocytosis of existing crystals may result in lactic acid generation, thus lowering the synovial fluid pH and promoting additional local impetus for crystal formation. Consistent with this model, increasing lactic acid and declining pH have been observed in the synovial fluid of acute gout. In addition, it has been postulated that the increased incidence of acute gouty attacks that occurs during sleep may be due in part to the mild respiratory acidosis that ensues when breathing rates decline. As such, the disclosure provides a solution to raise bodily fluid, e.g., synovial fluid, pH by the addition of an antacid or alkali, such as, e.g., sodium bicarbonate, which would effectively lower overall acidity and thus prevent or reverse hyperuricemia.

In addition to the above-mentioned factors, mechanical effects may also promote MSU crystallization. Physical perturbations of a supersaturated urate solution can directly induce the formation of MSU crystals. These observations may be consistent with the predilection for gout to occur in joints rather than other tissues, since joints undergo repeated daily mechanical shocks.

The delivery of treatment compounds to a patient is conventionally performed in a number of different ways, such as, e.g., intravenous delivery is into a blood vessel; intraperitoneal delivery is into the peritoneum; subcutaneous delivery is under the skin; intramuscular delivery is into a muscle tissue; and oral delivery is through the mouth.

An effective method for drug delivery is through the skin. Skin comprises the epidermis, including the stratum corneum, stratum granulosum, stratum spinosum, and stratum basale, and the dermis comprising the capillary layer. The stratum corneum is a tough, scaly layer made of dead cell tissue that extends around 10-20 microns from the skin surface and has no blood supply. It may have a thickness of 10-15 microns and is continually renewed by a keranization process.

Transdermal delivery systems and methods have been developed for the administration of pharmaceuticals at desired sustained levels by absorption through the skin. Typically, devices used in such techniques—often referred to as “patches”—are attached to the skin of a patient, usually adhesively. The active ingredient or agent is transported from the patch through the skin for absorption into the bloodstream. Upon absorption, the agent is carried throughout the body of the patient.

Transdermal delivery of drugs may allow one or more pharmaceutical agents to be introduced into a patient's system at a specific dosage and controlled rate. The drug may penetrate the skin surface by various passive or active mechanisms. Examples of passive mechanisms may include simple diffusion or absorption, such as, e.g., osmosis. Active mechanisms may include mechanical methods, such as, e.g. piercing and abrasion, or electrical methods, such as, e.g., iontophoresis. Other techniques, such as, e.g., the use of chemical penetration enhancers, anti-irritants, prodrugs, heat, mechanical perturbations, and/or ultrasound waves, may also be used to increase drug delivery rates through the skin barrier. For example, a penetration enhancer may be substance that is able to increase the permeability of a patient's skin to the active ingredient or agent. The patch may release one or more enhancers to the surface of the stratum corneum to modify the barrier properties of the skin, and may be incorporated into the layer or section including the active ingredient or agent, such as, e.g., adhesive layer, matrix, and/or reservoir. Example enhancers include fatty acids, fatty alcohols, alkyl methyl sulfoxides, pyrrolidones, glycols, and azocyclo-alkan-2-ones.

Systems and methods for the use of a transdermal patch for preventing, minimizing or reversing hyperuricemia, are presented. Transdermal application of sodium bicarbonate avoids the chemically hostile gastrointestinal tract environment, such as, e.g., reaction with stomache acids that may neutralize the compound prior to entering the circulatory system, as may be observed in an oral application. Upon removal of a peel strip, the patch may be adhered to the skin of a patient at a desirable location, such as, e.g., an affected joint. The active ingredient or agent, e.g. antacid or alkali, may be distributed in the patch in any suitable manner. For example, a specified amount of the active ingredient or agent may be contained in a single drug-containing layer or multiple drug-containing layers. In addition to the active ingredient or agent, the drug-containing layer may comprise a carrier material for carrying the active ingredient or agent. The active ingredient or agent may be mixed with the carrier material, such as, e.g. homogenously blended, heterogeneously dispersed, encapsulated within, or any combination thereof.

In general, the carrier material comprises ingredients that are suitable for transdermal drug delivery, such as, e.g., adhesives, liquids, solvents, solubizers, additives, adjuvants, plasticizers, tackifiers, skin penetration enhancers, crosslinking agents, and/or other substances. For example, sodium bicarbonate may be mixed with an organic solvent, e.g., alcohols and glycols, or aqueous solvent, e.g., water, or a mixture thereof, either prior to packaging or configured to mix prior to application, such as a patch comprising a breakable compartment that releases the solvent onto the sodium bicarbonate. The organic solvent may be a polar organic solvent, as the active ingredient or agent may be more soluble in polar solvents than in non-polar solvents. The polar organic solvent may be protic or aprotic. Examples of polar aprotic organic solvents include N-methylpyrrolidone, tetrahydrofuran, ethyl acetate, acetone, dimethylformamide, acetonitrile, dimethyl sulfoxide, dichloromethane, dimethylformamide, and hexamethylphosphoric triamide. Examples of polar protic organic solvents include formic acid, n-propanol, n-butanol, t-butanol, isopropanol, nitromethane, ethanol, methanol, and acetic acid. Examples of carrier materials that are non-reactive to sodium bicarbonate include hydrophobic hydrocarbons, e.g., petroleum gelly, and gelling agents, e.g., hydroxyethyl cellulose, hydroxypropyl cellulose, polyvinylpyrrolidone, and agar. An inert carrier material that has a melting point above room temperature, e.g., 20-22° C. —and below body temperature, e.g., 37° C.—such that it stays in a solid state during storage, e.g., at room temperature, and melts into a liquid state during usage at body temperature may also be used with the active ingredient or agent, such as, e.g., petroleum jelly or coconut oil.

Generally, the amount of carrier material, e.g., solvent, is the smallest necessary amount capable of dissolving or suspending the active ingredient. On the other hand, the concentration of active ingredient is the largest amount possible to be dissolved or distributed in the carrier material. The rate of percutaneous absorption can be affected by the oil/water partition coefficient, the polarity of the active ingredient or agent and its degree of ionization, its solubility characteristic, molecular weight, volatility, and concentration. A desired delivery rate can be achieved by varying the selection of ingredients, the amount of the ingredients, how the formulation is made, and other formulation process parameters.

Water and/or carriers comprising emollient properties, such as, e.g., glycols like glycerin, viscosity controlling agents, preservatives, thickening agents, antibacterial agents such as a quaternary ammonium compound, stabilizers, depletion indicating devices such as dyes, waxes and other material typically employed in pharmaceutical and cosmetic applications and which are dermatologically and pharmaceutically acceptable, may be used to hydrate and soften the stratum corneum to promote dermal absorption of the active ingredient or agent.

The transdermal patch may include any of a variety of design types, including but not limited to drug-in-adhesive, reservoir, matrix and multi-laminate. Drug-in-adhesive patches may compise an adhesive layer containing the active ingredient or agent. In this type of patch, the adhesive layer not only serves to hold the various layers intact while adhering to the skin, but it is also responsible for storing and releasing the active ingredient or agent. The adhesive layer may be surrounded by a temporary liner and a backing. The reservoir patch may comprise a reservoir separated from the skin by an inert polymeric membrane that controls the rate at which the drug is delivered. These patches offer a benefit that as long as the active ingredient or agent solution in the reservoir remains saturated, the drug release rate through the membrane is constant. The matrix patch is also known as a monolithic device. The matrix system incorporates a backing layer, a matrix layer, and an adhesive layer. The matrix layer is made of a polymer material in which the active ingredient or agent is distributed, and the rate at which the drug is released from the device is controlled by the polymer matrix. With this type of system, the release rate of active ingredient or agent falls off with time as the active ingredient or agent in the skin-contacting side of the matrix is depleted. Lastly, the multi-laminate patch may include two or more adhesive layers containing the active ingredient or agent with different release times. A bolus dose may initially deploy, followed by a maintenance dose. The active ingredients or agents in each of the adhesive layers may be the same or different.

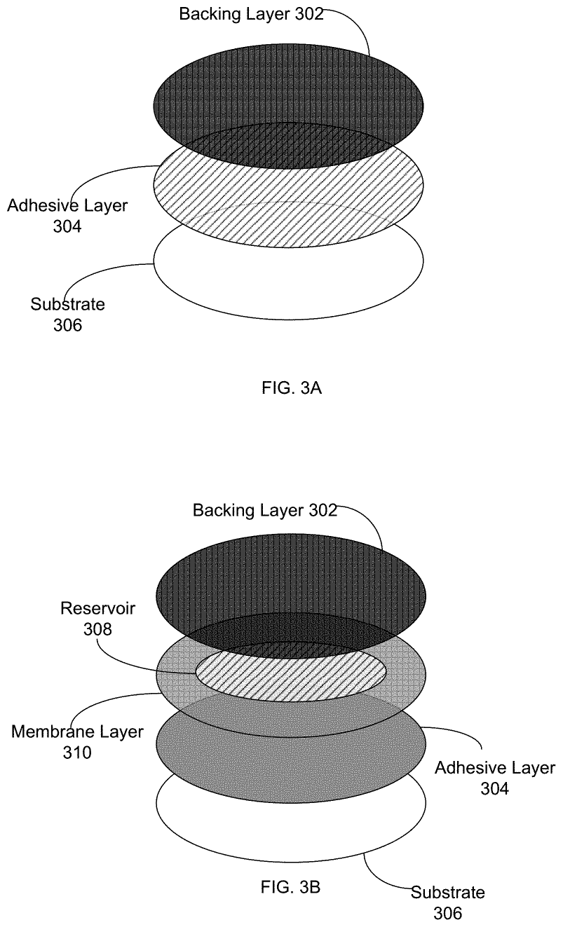

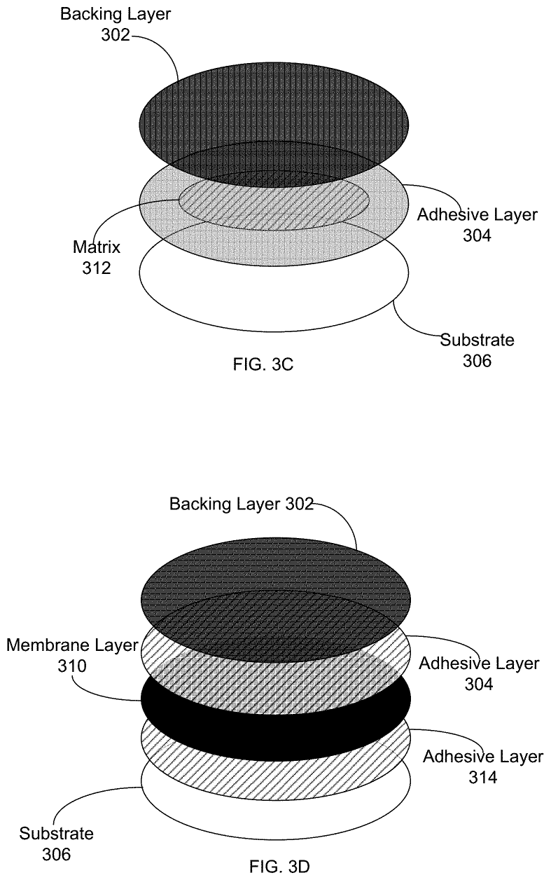

FIGS. 3A-D show different configurations for a transdermal patch of the present invention. An antacid or alkali, such as, e.g., sodium bicarbonate, may be the active ingredient or agent in each variation of the transdermal patch. An inert carrier material that has a melting point above room temperature, e.g., 20-22° C.—and below body temperature, e.g., 37° C.—such that it stays in a solid state during storage, e.g., at room temperature, and melts into a liquid state during usage at body temperature may be used with the active ingredient or agent, such as, e.g., petroleum jelly or coconut oil. Additionally, a compound, such as, e.g., capsaicin, may be included with the formulation to provide a warming sensation that may facilitate the opening of pores for efficient transdermal delivery.

In FIG. 3A, a drug-in-adhesive patch comprises backing layer 302, adhesive layer 304, and substrate 306. Backing layer 302 may be the outermost layer of the patch, which supports adhesive layer 304 and protects the formulation during the wear period. The material used for backing layer 302 may be occlusive, inert, impermeable, and/or incapable of absorbing the active ingredient or agent, or any other components of the formulation contained within, and may permit the patch to follow the contours of the skin and be worn comfortably on areas such as, e.g., joints or other points of flexure. The occlusion entraps sweat which may in turn serves to hydrate the skin, thus facilitating drug penetration. Example materials include woven and non-woven fabric, paper, plastic film, metal foil, polyethylene terephthalate (PET), polyethylene, polycarbonate, polypropylene, polybutadiene, polyvinyl chloride, polyether amide, polyester, nylon, cellophane, or polyurethane, or any combination thereof.

An active ingredient or agent may be distributed directly within adhesive layer 304, which may then simultaneously maintain the patch in close contact with substrate 306, and acts as a reservoir for the active ingredient or agent and as a modulator for its release. The adhesive may be provided in any suitable form, such as, e.g., a solid form, namely, powder, granules, and/or pellets, or mixed into an aqueous or organic solvent, namely, as a solution, dispersion, or emulsion. Examples of adhesive materials include acrylics, natural and synthetic rubbers, ethylene vinyl acetate, poly(alpha-olefins), vinyl ethers, polybutadiene, polyethylenes, polysiloxanes, polyisobutylnes, polyurethanes, neoprene, silicones, copolymers and mixtures thereof. Alternatively, a double-adhesive-coated medical tape may be used. One or more adhesive may be mixed with the active ingredient or agent homogenously or heterogeneously in any suitable amount. The chosen adhesive should not interact with the active ingredient or agent. In addition, adhesive layer 304 may further include an additional additive, such as, e.g., an additional medicinal ingredient or agent, a tackifier, a cross-linking agent, a softener or plasticizer, an absorpotion enhancer, polyhydric alcohols, silicone oils, an inorganic filler, and an ultraviolet absorber.

Substrate 306 may include a user or hyperuricemic patient's skin, or a release liner which protects adhesive layer 304 during storage and is removed prior to application of the patch to the patient's skin. The release liner does not absorb, and is impermeable to, components of the adhesive layer 304, including the active ingredient or agent, and may be formed from any suitable material, such as, e.g., polyethylene terephthalate (PET), fluorocarbon, polyester, polyurethane, polyvinyl acetate, polyvinylidene chloride, polypropylene, polycarbonate, polystyrene, polytetrafluroethylene polyethylene, cellophane, polyvinyl chloride, nylon, styrene, paper, non-woven fabric, woven fabric, metal foil, or laminate, or any combination thereof. The release liner may be siliconized and/or coated with fluoropolymer.

In FIG. 3B, a reservoir patch comprises a backing layer 302, adhesive layer 304, substrate 306, reservoir 308, and membrane layer 310. Backing layer 302 may be the outermost layer of the patch, which protects the formulation during the wear period. The material used for backing layer 302 may be occlusive, inert, impermeable, and/or incapable of absorbing the active ingredient or agent, or any other components of the formulation contained within, and may permit the patch to follow the contours of the skin and be worn comfortably on areas such as, e.g., joints or other points of flexure. The occlusion entraps sweat which may in turn serves to hydrate the skin, thus facilitating drug penetration. Example materials include woven and non-woven fabric, paper, plastic film, metal foil, polyethylene terephthalate (PET), polyethylene, polycarbonate, polypropylene, polybutadiene, polyvinyl chloride, polyether amide, polyester, nylon, cellophane, or polyurethane, or any combination thereof.

Adhesive layer 304 may maintain the patch in close contact with substrate 306. The adhesive may be provided in any suitable form, such as, e.g., a solid form, namely, powder, granules, pellets, and etc., or mixed into an aqueous or organic solvent, namely, as a solution, dispersion, or emulsion. Examples of adhesive materials include acrylics, natural and synthetic rubbers, ethylene vinyl acetate, poly(alpha-olefins), vinyl ethers, polybutadiene, polyethylenes, polysiloxanes, polyisobutylnes, polyurethanes, neoprene, silicones, copolymers and mixtures thereof. Alternatively, a double-adhesive-coated medical tape may be used. One or more adhesive may be mixed with the active ingredient or agent homogenously or heterogeneously in any suitable amount. The chosen adhesive should not interact with the active ingredient or agent. In addition, adhesive layer 304 may further include an additional additive, such as, e.g., an additional medicinal ingredient or agent, a tackifier, a cross-linking agent, a softener or plasticizer, an absorpotion enhancer, polyhydric alcohols, silicone oils, an inorganic filler, and an ultraviolet absorber.

Substrate 306 may include a user or hyperuricemic patient's skin, or a release liner which protects adhesive layer 304 during storage and is removed prior to application of the patch. The release liner does not absorb, and is impermeable to, components of the reservoir 308 and/or membrane layer 310, including the active ingredient or agent, and may be formed from any suitable material, such as, e.g., polyethylene terephthalate (PET), fluorocarbon, polyester, polyurethane, polyvinyl acetate, polyvinylidene chloride, polypropylene, polycarbonate, polystyrene, polytetrafluroethylene polyethylene, cellophane, polyvinyl chloride, nylon, styrene, paper, non-woven fabric, woven fabric, metal foil, or laminate, or any combination thereof. The release liner may be siliconized and/or coated with fluoropolymer.

Reservoir 308 may be a compartment comprising, e.g., an open volume space, a viscous liquid, a gel, a solid structure, or a porous polymer, containing the active ingredient or agent and may be configured for controlled release through the patient's skin. Alternatively, reservoir 308 may be configured to receive a biological fluid sample from the patient's skin, such as, e.g., blood serum and sweat. Example materials include gum, e.g., guar, acacia, xanthan, a gel, or polymer, e.g, carboxypolymethylene, hydroxyethylcellulose or polyacrylamide. Reservoir 308 may include a topical formulation, such as, e.g., a gel, ointment, or lotion. A flexible layer of adsorbent material may be used to give structure to reservoir 308, such as a woven or nonwoven fabric, e.g., polyester, polyethylene, polypropylene or polyamides. Water may be present to enhance flux of the active ingredient or agent through the skin. Thus, reservoir 308 may be aqueous, e.g., contain water, or may be nonaqueous and used in combination with occlusive backing layer 302. In some cases, however, such as with an occlusive gel, a nonaqueous formulation may be used with or without occlusion of backing layer 302. The reservoir may comprise a breakable compartment that serves to separate the water or other carrier material from the active ingredient or agent during storage.

Membrane layer 310 may comprise a permeable, semi-permeable, microporous, and/or macroporous material that control the rate of compound diffusion out of the patch and into the skin. Microporous membranes used may be selected based on desired flux of one or more components, e.g., active ingredient or agent, contained in the formulation. A macroporous membrane, on the other hand, may control amount and thickness of the active ingredient or agent in contact with the skin by permitting the material in reservoir 308 to exude through the macropores to form a thin film at the point of contact. The thin film may comprise water to hydrate the skin by being in simultaneous contact with the stratum corneum and reservoir 308, thus increasing diffusion rate of the active ingredient or agent.

In FIG. 3C, a matrix patch comprises a backing layer 302, adhesive layer 304, substrate 306, and matrix 312. Backing layer 302 may be the outermost layer of the patch, which protects the formulation during the wear period. The material used for backing layer 302 may be occlusive, inert, impermeable, and/or incapable of absorbing the active ingredient or agent, or any other components of the formulation contained within, and may permit the patch to follow the contours of the skin and be worn comfortably on areas such as, e.g., joints or other points of flexure. The occlusion entraps sweat which may in turn serves to hydrate the skin, thus facilitating drug penetration. Example materials include woven and non-woven fabric, paper, plastic film, metal foil, polyethylene terephthalate (PET), polyethylene, polycarbonate, polypropylene, polybutadiene, polyvinyl chloride, polyether amide, polyester, nylon, cellophane, or polyurethane, or any combination thereof.

Adhesive layer 304 may maintain the patch in close contact with substrate 306. The adhesive may be provided in any suitable form, such as, e.g., a solid form, namely, powder, granules, pellets, and etc., or mixed into an aqueous or organic solvent, namely, as a solution, dispersion, or emulsion. Examples of adhesive materials include acrylics, natural and synthetic rubbers, ethylene vinyl acetate, poly(alpha-olefins), vinyl ethers, polybutadiene, polyethylenes, polysiloxanes, polyisobutylnes, polyurethanes, neoprene, silicones, copolymers and mixtures thereof. Alternatively, a double-adhesive-coated medical tape may be used. One or more adhesive may be mixed with the active ingredient or agent homogenously or heterogeneously in any suitable amount. The chosen adhesive should not interact with the active ingredient or agent. In addition, adhesive layer _04 may further include an additional additive, such as, e.g., an additional medicinal ingredient or agent, a tackifier, a cross-linking agent, a softener or plasticizer, an absorpotion enhancer, polyhydric alcohols, silicone oils, an inorganic filler, and an ultraviolet absorber.

Substrate 306 may include a user or hyperuricemic patient's skin, or a release liner which protects adhesive layer 304 during storage and is removed prior to application of the patch. The release liner does not absorb, and is impermeable to, components of adhesive layer 304 and/or matrix 312, including the active ingredient or agent, and may be formed from any suitable material, such as, e.g., polyethylene terephthalate (PET), fluorocarbon, polyester, polyurethane, polyvinyl acetate, polyvinylidene chloride, polypropylene, polycarbonate, polystyrene, polytetrafluroethylene polyethylene, cellophane, polyvinyl chloride, nylon, styrene, paper, non-woven fabric, woven fabric, metal foil, or laminate, or any combination thereof. The release liner may be siliconized and/or coated with fluoropolymer.

Adhesive layer 304 may form a peripheral ring around the outer margin of matrix 312, and may adhere to the user or hyperuricemic patient's skin. Alternatively, adhesive layer 304 and matrix 312 may be separate and distinct layers, with matrix 312 underlying adhesive layer 304. Matrix 312 may comprise two surfaces: a first surface and a second surface opposite the first surface. The first surface may be in contact with backing layer 302 and the second surface comprising the active ingredient or agent may be in contact substrate 306. The active ingredient or agent may migrate from the matrix, through the skin, and into the blood stream of the user or hyperuricemic patient.

Selection of material for matrix 312 may depend on the solubility and diffusivity of the active ingredient or agent and the time frame during which release is sought. For example, matrix 312 may be permeable, impermeable or semi-permeable to the active ingredient or agent, and may include e.g., porous woven or nonwoven fiber webs, apertured films, foams, sponges, gums, and gelling agents. Polymeric materials may also be used to form the matrix, such as, e.g., silicones, acrylic resins, acetate copolymers, plasticized polyvinyl acetate/polyvinyl chloride resins, plasticized hydrolyzed polyvinyl alcohol, rubber-based adhesives, plasticized polyvinyl chloride, polyethylene, polypropylenes, cellulose derivatives, polyurethanes, polysulfones, polyisobutylenes, polystyrenes, silica, gelatins and polyolefins. Matrix 312 may contain, or be treated with, any of the excipients or reagents that reservoir 308 of FIG. 3B comprises.

In FIG. 3D, a multi-laminate patch may resemble a drug-in-adhesive patch and may comprise a backing layer 302, adhesive layer 304, substrate 306, additional adhesive layer 314, and membrane layer 310. Backing layer 302 may be the outermost layer of the patch, which protects the formulation during the wear period. The material used for backing layer 302 may be occlusive, inert, impermeable, and/or incapable of absorbing the active ingredient or agent, or any other components of the formulation contained within, and may permit the patch to follow the contours of the skin and be worn comfortably on areas such as, e.g., joints or other points of flexure. The occlusion entraps sweat which may in turn serves to hydrate the skin, thus facilitating drug penetration. Example materials include woven and non-woven fabric, paper, plastic film, metal foil, polyethylene terephthalate (PET), polyethylene, polycarbonate, polypropylene, polybutadiene, polyvinyl chloride, polyether amide, polyester, nylon, cellophane, or polyurethane, or any combination thereof.

An active ingredient or agent may be distributed directly within adhesive layer 304, which may act as a reservoir for the active ingredient or agent and as a modulator for its release. The adhesive may be provided in any suitable form, such as, e.g., a solid form, namely, powder, granules, pellets, and etc., or mixed into an aqueous or organic solvent, namely, as a solution, dispersion, or emulsion. Examples of adhesive materials include acrylics, natural and synthetic rubbers, ethylene vinyl acetate, poly(alpha-olefins), vinyl ethers, polybutadiene, polyethylenes, polysiloxanes, polyisobutylnes, polyurethanes, neoprene, silicones, copolymers and mixtures thereof. Alternatively, a double-adhesive-coated medical tape may be used. One or more adhesive may be mixed with the active ingredient or agent homogenously or heterogeneously in any suitable amount. The chosen adhesive should not interact with the active ingredient or agent. In addition, adhesive layer 304 may further include an additional additive, such as, e.g., an additional medicinal ingredient or agent, a tackifier, a cross-linking agent, a softener or plasticizer, an absorpotion enhancer, polyhydric alcohols, silicone oils, an inorganic filler, and an ultraviolet absorber.

Substrate 306 may include a user or hyperuricemic patient's skin, or a release liner which protects adhesive layer 304 during storage and is removed prior to application of the patch. The release liner does not absorb, and is impermeable to, components of the adhesive layer 314 and/or adhesive layer 304, including the one or more active ingredient or agent, and may be formed from any suitable material, such as, e.g., polyethylene terephthalate (PET), fluorocarbon, polyester, polyurethane, polyvinyl acetate, polyvinylidene chloride, polypropylene, polycarbonate, polystyrene, polytetrafluroethylene polyethylene, cellophane, polyvinyl chloride, nylon, styrene, paper, non-woven fabric, woven fabric, metal foil, or laminate, or any combination thereof. The release liner may be siliconized and/or coated with fluoropolymer.

Additional adhesive layer 314 may also comprise an active ingredient or agent distributed directly within, which may then simultaneously maintain the patch in close contact with substrate 306, and acts as a reservoir for the active ingredient or agent and as a modulator for its release. The multi-laminate patch may be configured for immediate release of the active ingredient or agent within additional adhesive layer 314 followed by controlled release of the active ingredient or agent within adhesive layer 304. Adhesive layer 304 and additional adhesive layer 314 may comprise the same adhesive, or different adhesives. For example, adhesive layer 304 may comprise a silicone adhesive, and additional adhesive layer 314 may comprise an acrylic adhesive. The active ingredient or agent in adhesive layer 304 and additional adhesive layer 314 may also be the same or different. For example, adhesive layer 304 may comprise sodium bicarbonate, and additional adhesive layer 314 may comprise calcium carbonate.

Membrane layer 310 may comprise a permeable, semi-permeable, microporous, and/or macroporous material that separate adhesive layer 304 and additional adhesive layer 314, and may control the release of the active ingredient or agent within additional adhesive layer 314. Membranes used may be selected based on desired flux of one or more components, e.g., active ingredient or agent, contained in the formulation.

Any suitable manufacturing technique could be used to make the transdermal patch. For example, the pressure-sensitive adhesive layer in a drug-in-adhesive patch may be formed by, e.g., a solvent-casting method, a spray-coating method, an emulsion method, a heat-melting method, and an electron beam curing method.

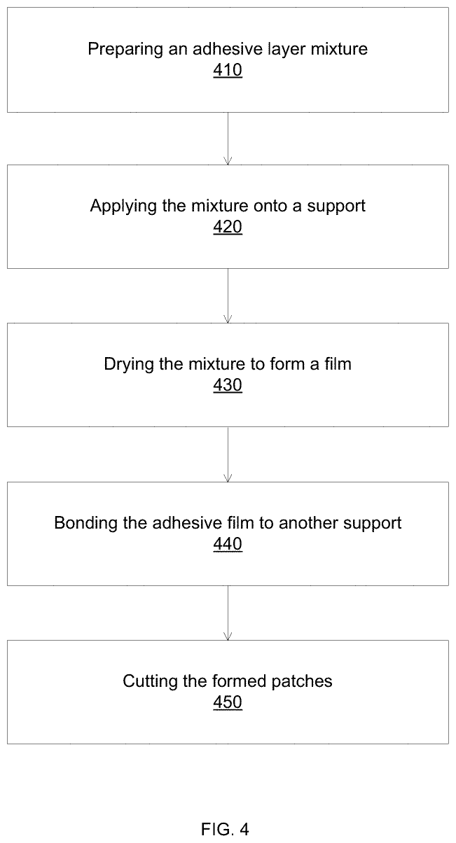

FIG. 4 is a flowchart of an example method for manufacturing a drug-in-adhesive patch. Operation 410 prepares an adhesive layer mixture containing an adhesive and an active ingredient or agent, such as, e.g., an antacid or alkali. Other compounds may be included in a mixture, such as, e.g., an additional medicinal ingredient or agent, a tackifier, a cross-linking agent, a softener or plasticizer, an absorpotion enhancer, polyhydric alcohols, silicone oils, an inorganic filler, and an ultraviolet absorber. The components may be combined at a suitable temperature in any suitable way. For example, in the solvent-casting method, the adhesive may be provided in a solvent liquid, such as, e.g., an organic solvent and/or an aqueous solvent, and the active ingredient or agent may be combined into the adhesive-solvent mixture. In another example, the components may be provided separately and the adhesive is mixed into the solvent liquid and then the active ingredient or agent is added to the mixture. In yet another example, the adhesive may be made by polymerization of monomers in a solvent liquid, and then the active ingredient or agent may be added to the polymerized adhesive. In each of the aforementioned examples, the mixture may be homogenously blended or heterogeneously distributed.

In the heat melting method, a drug mixture of the active ingredient or agent and a adhesive compound may be melted using heat, such as, e.g., at room temperature or artificially raised above room temperature. For example, the adhesive compound may be melted first and then the active ingredient or agent is added. Alternatively, the adhesive compound may be mixed together with the active ingredient or agent along with other compounds, and then the mixture is heat-melted.

Operation 420 applies the mixture containing the active ingredient or agent, e.g. by casting, onto a support, such as, e.g., a release liner or a backing layer. A spreading tool may be used to spread or level the mixture, such as, e.g., a film application tool, a roller, and/or a spreading knife. Operation 430 dries the solvents within the mixture to form a film. This can be done at room temperature with ambient air, forced air, such as, e.g., use of a fan, in a heated environment, such as, e.g., within an oven, and/or in a chilled environment, such as, e.g., within a refrigeration device. The thickness of the film, and thus the adhesive layer, is determined primarily by the distance of the tool or knife used to apply the mixture, if one is used at all. Operation 440 bonds the exposed side of the film to another support, such as, e.g., a release liner or a backing layer. For example, if a release liner was initially casted upon with the mixture containing the active ingredient or agent to form the film, then the exposed side of the film would be bonded, e.g., laminated, to a backing layer. The opposite may be true, wherein if a backing layer was initially casted upon with the mixture containing the active ingredient or agent to form the film, then the exposed side of the film would be bonded, e.g., laminated, to a release liner. Operation 450 cuts the formed patches into desired shapes and dimensions.

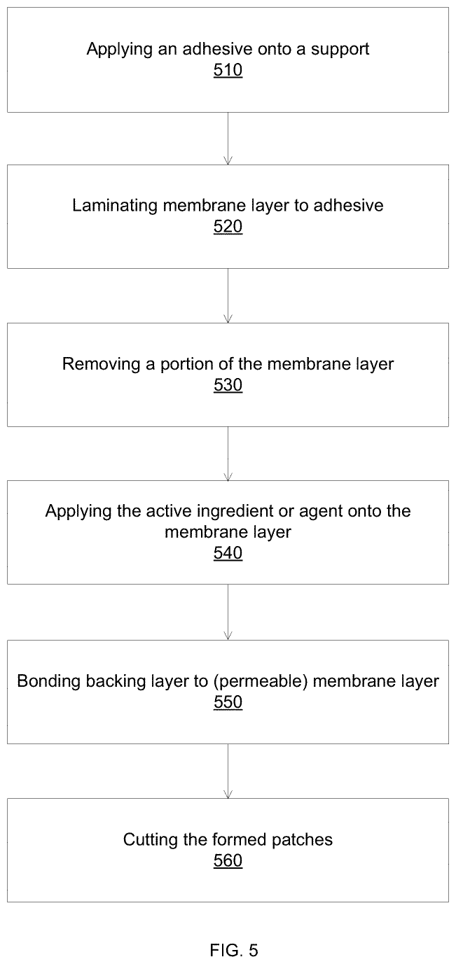

FIG. 5 is a flowchart of an example method for manufacturing a reservoir patch.

Operation 510 applies an adhesive, e.g. by casting, onto a support, such as, e.g., a release liner or a backing layer. A spreading tool may be used to spread or level the mixture, such as, e.g., a film application tool, a roller, and/or a spreading knife. Operation 520 laminates, a membrane layer to the adhesive. The membrane layer may be permeable. In some cases, it is be pre-made. Operation 530 removes a portion of the membrane layer, such as, e.g., cutting the membrane layer into a closed shape and portions of the membrane and adhesive layer that are peripheral to the closed shape are removed. Operation 540 applies the active ingredient or agent onto the membrane layer. Operation 550 bonds the backing layer to the membrane layer to form an enclosed reservoir containing the active ingredient or agent. In some cases, the bonding of the backing layer to the membrane layer includes heat-sealing. The backing layer may be sealed such that a portion of the backing layer extends beyond the outer perimeter of the membrane layer. Operation 560 cuts the formed patches into desired shapes and dimensions.

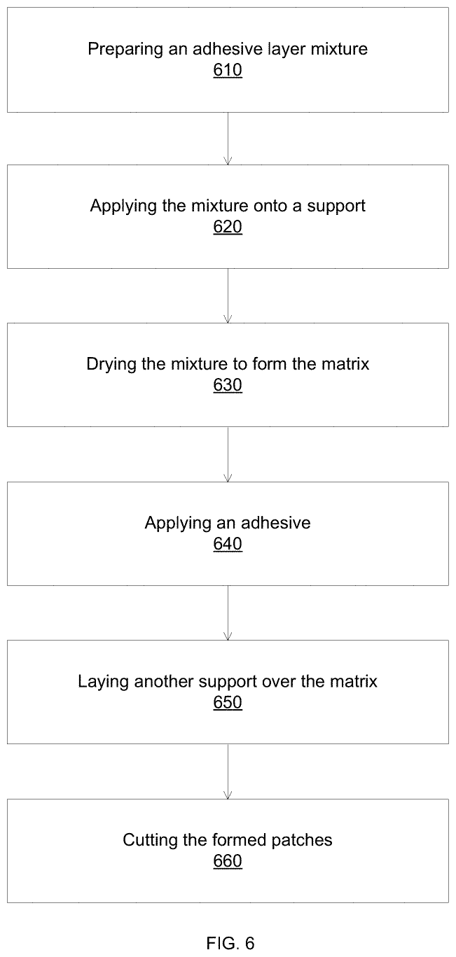

FIG. 6 is a flowchart of an example method for manufacturing a matrix patch. Operation 610 prepares a matrix mixture containing an active ingredient or agent, such as, e.g, an antacid or alkali, in addition to one or more other compounds, such as, e.g., an additional medicinal ingredient or agent, a tackifier, a cross-linking agent, a softener or plasticizer, an absorpotion enhancer, polyhydric alcohols, silicone oils, an inorganic filler, and an ultraviolet absorber. The components may be combined at a suitable temperature in any suitable way. For example, the components may be provided separately and mixed into a solvent solution, such as, e.g., an organic solvent and/or an aqueous solvent, and then the active ingredient or agent is added to the mixture. The mixture may be homogenously blended or heterogeneously distributed. In some cases, such as, e.g., large scale manufacturing, the matrix mixture may result in a homogeneous molten mass. Operation 620 applies the mixture onto a support, such as, e.g, a backing layer. Operation 630 dries the mixture to form the resulting matrix. This can be done at room temperature with ambient air, forced air, such as, e.g., use of a fan, in a heated environment, such as, e.g., within an oven, and/or in a chilled environment, such as, e.g., within a refrigeration device. Operation 640 applies an adhesive film, such as, e.g., by coating the edges of the backing layer, or double-sided medical adhesive tape is attached. Operation 650 lays another support, such as, e.g., a release liner, over the matrix and adhesive rim. Operation 660 cuts the formed patches into desired shapes and dimensions.

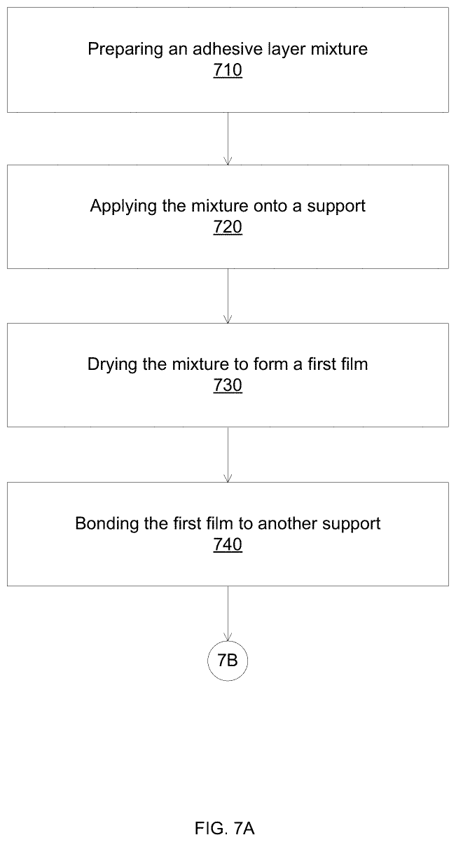

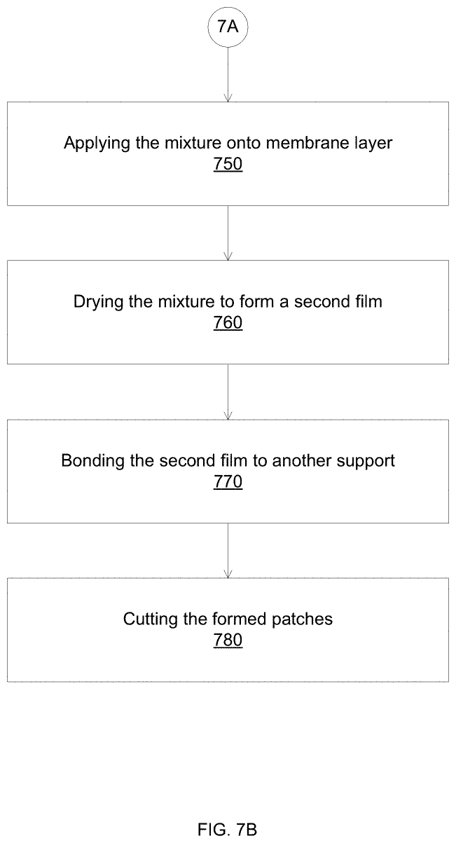

FIGS. 7A-B are flowcharts of an example method for manufacturing a multilaminate patch. Operation 710 prepares an adhesive layer mixture containing an adhesive and an active ingredient or agent, such as, e.g., an antacid or alkali. Other compounds may be included in a mixture, such as, e.g., an additional medicinal ingredient or agent, a tackifier, a cross-linking agent, a softener or plasticizer, an absorpotion enhancer, polyhydric alcohols, silicone oils, an inorganic filler, and an ultraviolet absorber. The components may be combined at a suitable temperature in any suitable way. For example, in the solvent-casting method, the adhesive may be provided in a solvent liquid, such as, e.g., an organic solvent and/or an aqueous solvent, and the active ingredient or agent may be combined into the adhesive-solvent mixture. In another example, the components may be provided separately and the adhesive is mixed into the solvent liquid and then the active ingredient or agent is added to the mixture. In yet another example, the adhesive may be made by polymerization of monomers in a solvent liquid, and then the active ingredient or agent may be added to the polymerized adhesive. In each of the aforementioned examples, the mixture may be homogenously blended or heterogeneously distributed.

In the heat melting method, a drug mixture of the active ingredient or agent and a adhesive compound may be melted using heat, such as, e.g., at room temperature or artificially raised above room temperature. For example, the adhesive compound may be melted first and then the active ingredient or agent is added. Alternatively, the adhesive compound may be mixed together with the active ingredient or agent along with other compounds, and then the mixture is heat-melted.

Operation 720 applies the mixture containing the active ingredient or agent, e.g. by casting, onto a support, such as, e.g., a release liner or a backing layer. A spreading tool may be used to spread or level the mixture, such as, e.g., a film application tool, a roller, and/or a spreading knife. Operation 730 dries the solvents within the mixture to form a first film. This can be done at room temperature with ambient air, forced air, such as, e.g., use of a fan, in a heated environment, such as, e.g., within an oven, and/or in a chilled environment, such as, e.g., within a refrigeration device. The thickness of the film, and thus the adhesive layer, is determined primarily by the distance of the tool or knife used to apply the mixture, if one is used at all. Operation 740 bonds the exposed side of the first adhesive layer film to another support, such as, e.g., a membrane layer. Operation 750 applies the mixture containing the active ingredient or agent, adhesive, and other components onto the exposed side of the membrane layer. A different mixture may be used in the second adhesive layer, such as, e.g., a different active ingredient or agent. Operation 760 dries the solvents within the mixture to form a second film. This can be done at room temperature with ambient air, forced air, such as, e.g., use of a fan, in a heated environment, such as, e.g., within an oven, and/or in a chilled environment, such as, e.g., within a refrigeration device. The thickness of the film, and thus the adhesive layer, is determined primarily by the distance of the tool or knife used to apply the mixture, if one is used at all. Operation 770 bonds the exposed side of the second adhesive layer film. Operation 780 cuts the formed patches into desired shapes and dimensions.

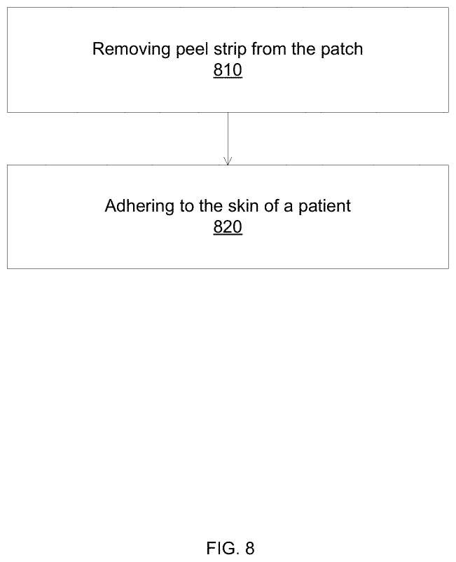

FIG. 8 is a flowchart of a method for using the transdermal patch. Operation 810 removes a peel strip from the patch upon removal from the original packaging. Operation 820 adheres the patch to the skin of a hyperuricemic patient at a desirable location, such as, e.g., an affected joint. In some cases, applies a fresh transdermal patch within a predetermined time frame, such as, e.g, within minutes, hours, and days.

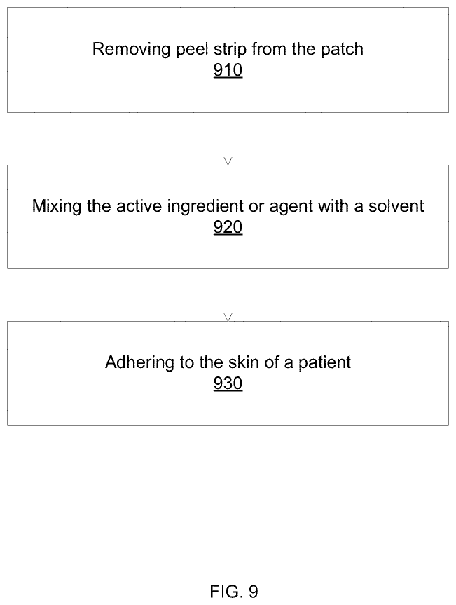

FIG. 9 is a flowchart of another method for using the transdermal patch. Operation 910 removes a peel strip from the patch upon removal from the original packaging. Operation 920 mixes the active ingredient or agent with a solvent, such as, e.g., an organic solvent, an aqueous solvent, or a mixture thereof. For example, to mix the active ingredient or agent with the solvent, a compartment that may be breakable upon physical exertion, such as, e.g. a bending motion, may be broken permitting the active ingredient or agent and the solvent to come into contact. Operation 930 adheres the patch to the skin of a hyperuricemic patient at a desirable location, such as, e.g., an affected joint. In some cases, a fresh transdermal patch is applied within a predetermined time frame, such as, e.g, minutes, hours, or days.

While the system and method of delivery of the active ingredient or agent may vary, they typically involve topical application of a formulation containing a pharmaceutically acceptable carrier to a predetermined area of the skin or other tissue for a period of time sufficient to provide the desired local or systemic effect, and may involve direct application of the composition as an ointment, gel, cream, lotion, solutions, pastes or the like, or may involve use of a drug delivery device such as a patch. Ointments are semi solid preparations that may be based on petrolatum or petroleum derivatives. The specific ointment foundation to be used, as will be appreciated by those skilled in the art, is one that will provide for optimum drug delivery, and, preferably, will provide for other desired characteristics, such as, e.g., emolliency or the like. As with other carriers or vehicles, the ointment foundation should be inert, stable, nonirritating and nonsensitizing.

Test subject “A” and control subject “B” took part in an experiment to verify and confirm that an antacid or alkali, and in this particular case, sodium bicarbonate, has the effect of preventing, mimimizing and/or reversing hyperuricemia in patients by raising the pH level of bodily fluids. The method of the experiment includes test subject A submerging an affected joint into a warm bath containing water and sodium bicarbonate. The heat was used to facilitate opening of skin pores to facilitate absorption of the active ingredient or agent. Sodium bicarbonate was added and mixed into water at 38° C. until it no longer dissolves, and hence saturation has been achieved. For reference, the solubility of sodium bicarbonate in water is 96 g/L at 20° C. Test subject A submerged their affected foot into the water bath solution for twice a day for durations of 60 minutes, and for a total of six months. Control subject B submerged their affected foot into a pure water bath without any other substance or compound at 38° C. for twice a day for durations of 60 minutes, and for a total of six months. Uric acid serum levels were measured on the 1^{st} and 15^{th} of each month for six consecutive months in the year 2018 at a health clinic. A phlebotomist drew a vial of blood from each subject's arm, and the samples were processed by an analyzer, model AU480, by Beckman Coulter. The results of the measurements are tabulated in Table 1 below.

Both test subject A and control subject B were diagnosed of hyperuricemia based on personal and clinical assessments, such as, e.g., multiple previous acute gout attacks, presence of swelling and tophi at the affected joint, and measured uric acid serum levels above 6.8 mg/L. Both test subject A and control subject B were not on any medication at the time of the experiment, and maintained a low-purine diet, such as, e.g., primarily fruits, vegetables, and bread.

TABLE 1

| Date (2018) | A (mg/dL) | B (mg/dL) |
|---|---|---|
| June 1 | 6.99 | 7.25 |
| June 15 | 7.15 | 7.31 |
| July 1 | 6.62 | 7.26 |
| July 15 | 6.58 | 7.21 |
| August 1 | 6.79 | 7.20 |
| August 15 | 6.47 | 7.25 |
| September 1 | 5.98 | 7.35 |
| September 15 | 5.59 | 7.40 |
| October 1 | 5.47 | 7.39 |
| October 15 | 5.40 | 7.42 |
| November 1 | 5.37 | 7.41 |
| November 15 | 5.34 | 7.42 |
| December 1 | 5.41 | 7.48 |
| December 15 | 5.20 | 7.49 |

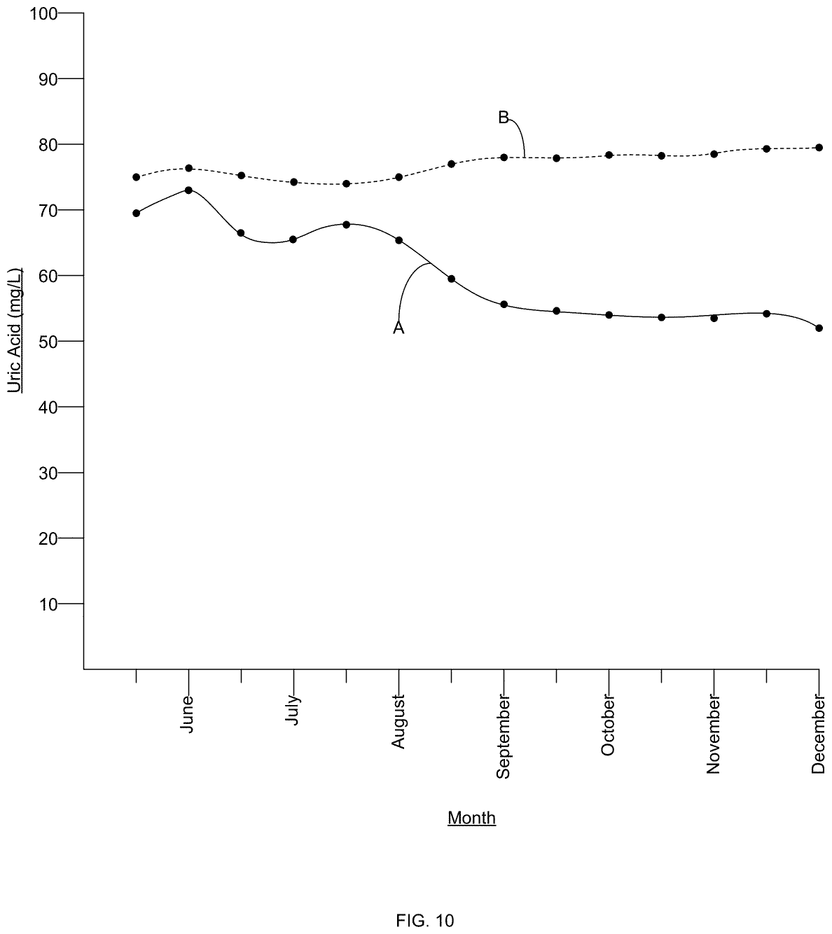

FIG. 10 is a graphical representation of Table 1. As can be ascertained, both test subject A and control subject B initially increased in uric acid content, followed by a decrease. Overall, test subject A experienced a general decline of uric acid content, which control subject B had a net increase. In addition, although size measurements were not taken on a scheduled basis, impact on tophi size of test subject A and control subject B appears to have profound difference over the six-month experiment. Specifically, test subject A's tophi was no longer visible by visual inspection while control subject B's tophi does not appear to be affected.

A number of examples have been described. Nevertheless, it will be understood that various modifications may be made without departing from the spirit and scope of the claimed invention. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other examples are within the scope of the following claims.

It may be appreciated that the various systems, methods, and apparatus disclosed herein may be configured in a machine-readable medium and/or a machine accessible medium, and/or may be performed in any order. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

What is claimed is:

1. (canceled)

2. (canceled)

3. A system, comprising:
an backing layer;
a drug-containing layer,
wherein the drug-containing layer comprises a base compound for treatment of hyperuricemia; and
a substrate.

4. The method of claim 3, further comprising:
wherein the base compound is sodium bicarbonate.

5. The method of claim 3, further comprising:
wherein the drug-containing layer comprises a reservoir juxtapose to the backing layer.

6. The method of claim 5, further comprising:
wherein the reservoir comprises an open space, a liquid, a gel, a solid structure, a porous membrane, or any combination thereof.

7. The method of claim 3, further comprising:
wherein the drug-containing layer comprises a polymer matrix coupled with an adhesive layer for adhering to a substrate, and
wherein the polymer matrix controls the rate of disbursement of the base compound.

8. The method of claim 3, further comprising:
a membrane layer disposed between the drug-containing layer and another drug-containing layer, and
wherein both drug-containing layers comprise an adhesive material

9. The method of claim 8, further comprising:
wherein the drug-containing layers comprise different adhesive materials, or wherein the drug-containing layers comprise the same adhesive material.

10. The method of claim 3, further comprising:
wherein the carrier material comprises a compound that is solid at ambient room temperature and liquid at body temperature.

11. A system, comprising:
an occlusive backing layer;
a drug-containing layer comprising an emollient carrier material;
wherein the drug-containing layer comprises a base compound for decreasing a blood serum acidity level,
wherein the occlusive backing layer is impermeable to the base compound;
a substrate,
wherein the substrate comprises a release liner or human skin.

12. The method of claim 11, further comprising:
wherein the carrier material is petroleum jelly, coconut oil, or both.

13. The method of claim 11, further comprising:
wherein the drug-containing layer comprises water for increasing transdermal penetration of the base compound.

14. The method of claim 11, further comprising:
wherein the drug-containing layer comprises a reservoir juxtapose to the backing layer,
wherein the reservoir comprises an open space, a liquid, a gel, a solid structure, a porous membrane, or any combination thereof, and
wherein the reservoir is coupled with a membrane layer for controlling a rate of disbursement of the base compound.

15. The method of claim 14, further comprising:
an adhesive layer coupled to the remaining side of the membrane layer for adhering to a substrate.

16. The method of claim 11, further comprising:
wherein the drug-containing layer comprises a penetration enhancer, an anti-irritant, or both

17. A system, comprising:
an occlusive backing layer;
a drug-containing layer comprising a carrier material;
wherein the drug-containing layer comprises sodium bicarbonate for decreasing a blood serum acidity level,
wherein the occlusive backing layer is impermeable to the base compound;
wherein the carrier material is the least amount capable of dissolving the base compound;
a release liner, and
wherein the release liner is impermeable to the base compound

18. The method of claim 17, further comprising:
a membrane layer disposed between the drug-containing layer and another drug-containing layer.

19. The method of claim 17, further comprising:
wherein the drug-containing layers comprise different base compounds, or wherein the drug-containing layers comprise the same base compound.

20. The method of claim 17, further comprising:
wherein the drug-containing layer comprises capsaicin.

21. The method of claim 17, further comprising:
wherein the drug-containing layer comprises an adhesive material.

22. The method of claim 17, further comprising:
wherein the drug-containing layer comprises a reservoir juxtapose to the backing layer, and
wherein the reservoir is coupled with a membrane layer for controlling a rate of disbursement of the base compound.
